(19) United States
(12) Patent Application Publication
(10) Pub. No.: US 2018/0185004 A1
(43) Pub. Date: Jul. 5, 2018

(54) ENDOSCOPE CONNECTOR

(71) Applicant: OLYMPUS CORPORATION, Tokyo (JP)

(72) Inventor: Kazuya SAIGA, Tokyo (JP)

(73) Assignee: OLYMPUS CORPORATION, Tokyo (JP)

(21) Appl. No.: 15/907,346

(22) Filed: Feb. 28, 2018

Related U.S. Application Data
(63) Continuation of application No. PCT/JP2016/061821, filed on Apr. 12, 2016.

(30) Foreign Application Priority Data
Sep. 2, 2015 (JP) 2015-172883

(51) Int. Cl. A61B 8/12

(52) U.S. Cl. CPC A61B 8/12; A61B 1/12

(57) ABSTRACT

An endoscope includes: an insertion unit adapted to be inserted into a subject; an operating unit; a universal cable whose first end is connected to the operating unit; and an endoscope connector connected to a second end of the universal cable, and extending along a first axis. The endoscope connector includes: a non-waterproof type ultrasound connector provided in a first region, extending along a second axis perpendicular to a virtual plane including the first axis, and electrically connected to an ultrasound observation device; a suction duct ferrule provided in a second region, and configured to be connected to a suction device that suctions fluid inside the subject; and an air/water supply duct ferrule provided in the second region, and configured to be connected to an air/water supply device that supplies air or water to an observation window of the insertion unit.

### CROSS-REFERENCE TO RELATED APPLICATIONS

This application is a continuation of PCT International Application No. PCT/JP2016/061821 filed on Apr. 12, 2016 which claims the benefit of priority from Japanese Patent Application No. 2015-172883, filed on Sep. 2, 2015, the entire contents of which are incorporated herein by reference.

### BACKGROUND

The present disclosure relates to an endoscope connector.

There have been known endoscope systems in which an ultrasound endoscope and an ultrasound observation device are connected to each other through an endoscope connector, the ultrasound endoscope including an insertion unit to be inserted into the inside of a subject, and the ultrasound observation device processing signals from an ultrasound probe provided at the distal end of the insertion unit (for example, see Japanese Laid-open Patent Publication No. 2000-166928).

The endoscope connector (scope connector unit) disclosed in Japanese Laid-open Patent Publication No. 2000-166928 is provided with an ultrasound connector, a suction duct ferrule (suction ferrule), an air/water supply duct ferrule (water supply ferrule), and the like.

The ultrasound connector is a connector that is connected to the ultrasound observation device in an attachable and removable manner and that transmits signals output from the ultrasound probe to the ultrasound observation device.

The suction duct ferrule is a ferrule for connecting to a suction device such as a suction pump. Furthermore, due to driving of the suction device, fluid or the like inside the subject is suctioned from the distal end of the insertion unit to the suction pump through the ultrasound endoscope and the endoscope connector (the suction duct ferrule).

The air/water supply duct ferrule is a ferrule for connecting to an air/water supply device such as an air supply pump or a water supply pump. Furthermore, due to driving of the air/water supply device, air or water is supplied to the outer surface of an observation window provided at the distal end of the insertion unit through the endoscope connector (the air/water supply duct ferrule) and the ultrasound endoscope.

### SUMMARY

An endoscope according to one aspect of the present disclosure includes: an insertion unit adapted to be inserted into a subject; an operating unit provided at a proximal end side of the insertion unit; a universal cable whose first end is connected to the operating unit; and an endoscope connector connected to a second end of the universal cable, and extending along a first axis, the endoscope connector including: a non-waterproof type ultrasound connector provided in a first region, extending along a second axis perpendicular to a virtual plane including the first axis, and electrically connected to an ultrasound observation device; a suction duct ferrule provided in a second region, and configured to be connected to a suction device for that suctions fluid inside the subject from a distal end of the insertion unit; and an air/water supply duct ferrule provided in the second region, and configured to be connected to an air/water supply device that supplies air or water to an observation window of the insertion unit, wherein the endoscope connector is virtually divided into the first region and the second region by the virtual plane including the first axis.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram that schematically illustrates an endoscope system according to an embodiment;

FIG. 2 is a perspective view when the endoscope connector illustrated in FIG. 1 is viewed from the front side and the upper left side;

FIG. 3 is a perspective view when the endoscope connector illustrated in FIG. 1 is viewed from the front side and the upper right side;

FIG. 4 is a diagram when the endoscope connector illustrated in FIG. 2 or 3 is viewed from the upper side;

FIG. 5 is a diagram when the endoscope connector illustrated in FIG. 2 or 3 is viewed from the lower side;

FIG. 6 is a diagram when the endoscope connector illustrated in FIG. 2 or 3 is viewed from the left side; and

FIG. 7 is a diagram when the endoscope connector illustrated in FIG. 2 or 3 is viewed from the right side.

### DETAILED DESCRIPTION

With reference to the drawings, an explanation is given below of an aspect (hereafter, embodiment) for implementing the present disclosure. Furthermore, the present disclosure is not limited to the embodiment described below. Moreover, for description in the drawings, the same parts are attached with the same reference numerals.

Schematic Configuration of Endoscope System

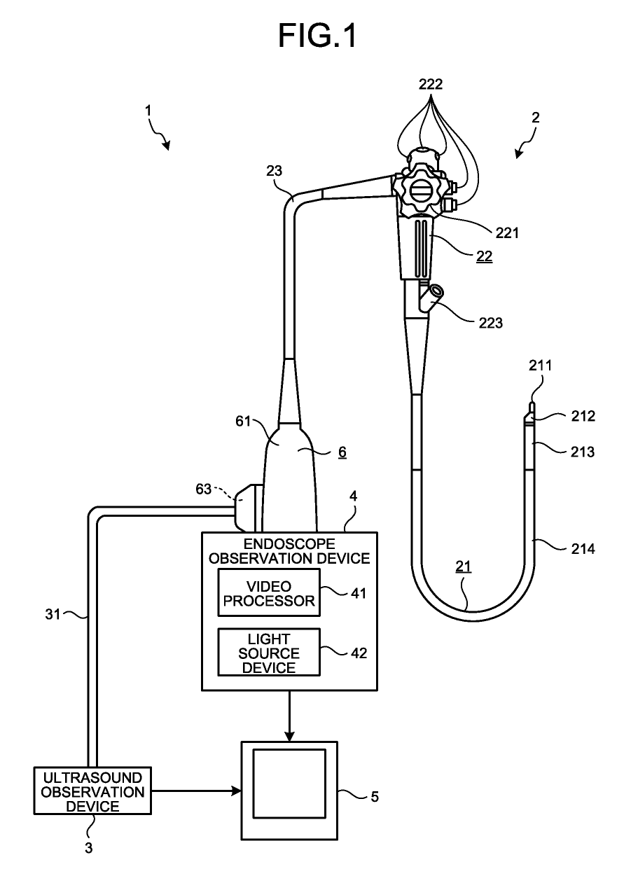

FIG. 1 is a diagram that schematically illustrates an endoscope system 1 according to an embodiment.

The endoscope system 1 is a system that conducts ultrasound diagnosis on the inside of subjects such as persons by using an ultrasound endoscope. As illustrated in FIG. 1, the endoscope system 1 includes an endoscope 2, an ultrasound observation device 3, an endoscope observation device 4, and a display device 5.

The endoscope 2 is an ultrasound endoscope that may be partially inserted into a subject such that it performs the function to transmit ultrasound pulses toward a body wall within the subject, receive ultrasound echoes reflected by the subject, and output echo signals and the function to capture the inside of the subject and output image signals.

Furthermore, the detailed configuration of the endoscope 2 is described later.

The ultrasound observation device 3 is electrically connected to the endoscope 2 via an ultrasound cable 31 (FIG. 1) so that it outputs pulse signals to the endoscope 2 via the ultrasound cable 31 and receives echo signals from the endoscope 2. Furthermore, the ultrasound observation device 3 performs predetermined processing on the echo signals to generate ultrasound images.

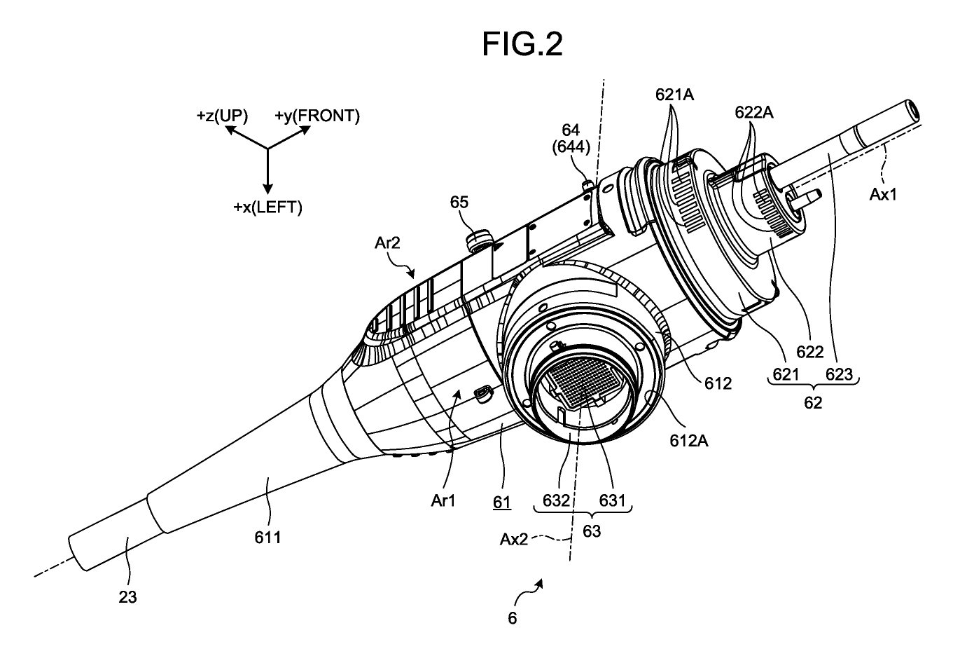

An endoscope connector 6 (see FIGS. 2 to 7), described later, of the endoscope 2 is connected to the endoscope observation device 4 in an attachable and removable manner. As illustrated in FIG. 1, the endoscope observation device 4 includes a video processor 41 and a light source device 42.

The video processor 41 receives image signals from the endoscope 2 via the endoscope connector 6. Furthermore, the video processor 41 performs predetermined processing on the image signals to generate endoscope images.

The light source device 42 supplies illumination light for lighting the inside of a subject to the endoscope 2 through the endoscope connector 6.

The display device 5 is formed by using liquid crystal or organic electro luminescence (EL), and it displays ultrasound images generated by the ultrasound observation device 3, endoscope images generated by the endoscope observation device 4, and the like.

Configuration of Endoscope

As illustrated in FIG. 1, the endoscope 2 includes an insertion unit 21, an operating unit 22, a universal cable 23, and the endoscope connector 6.

Here, although not illustrated in detail, a light guide that transmits illumination light supplied from the light source device 42, signal cables that transmits various types of signals, a suction tube, an air/water supply tube, and a front water-supply tube, or the like, are led to the inside of the endoscope 2 (the inside of the insertion unit 21, the operating unit 22, the universal cable 23, and the endoscope connector 6).

The insertion unit 21 is a part that is inserted into the inside of a subject. As illustrated in FIG. 1, the insertion unit 21 includes an ultrasound probe 211 that is provided on the distal end, a rigid member 212 that is connected to the proximal end side (the side of the operating unit 22) of the ultrasound probe 211, a curved portion 213 that is connected to the proximal end side of the rigid member 212 and that may be curved, and a flexible tube portion 214 that is connected to the proximal end side of the curved portion 213 and that has flexibility.

Here, although not illustrated in detail, an image guide for guiding optical images of the inside of a subject, a treatment tool tube for inserting various treatment tools (e.g., puncture needles), or the like, are led to the inside of the insertion unit 21 (the rigid member 212, the curved portion 213, and the flexible tube portion 214) in addition to the above-described light guide, the signal cables, the suction tube, the air/water supply tube, the front water-supply tube, or the like.

In the example illustrated in FIG. 1, the ultrasound probe 211 is a convex ultrasound probe, and it has a configuration such that multiple ultrasound transducers (not illustrated) are regularly arranged so as to form a convex circular arc.

Here, the ultrasound transducer includes an acoustic lens, a piezoelectric element, and a matching layer, and it acquires ultrasound echoes that contribute to ultrasound tomographic images inside a body wall of the subject.

Furthermore, the ultrasound probe 211 converts pulse signals, input from the ultrasound observation device 3 via the signal cables and the ultrasound cable 31, described above, into ultrasound pulses and transmits them to the inside of the subject. Furthermore, the ultrasound probe 211 converts ultrasound echoes reflected by the inside of the subject into electric echo signals and outputs them to the ultrasound observation device 3 via the signal cables and the ultrasound cable 31 that are described above.

The rigid member 212 is a rigid member that is made of resin material, and it has substantially a cylindrical shape.

Here, although not illustrated in detail, an observation window, an illumination window, a treatment tool path, a suction path, an air/water supply path, a front water-supply path, or the like, are formed in the rigid member 212.

The observation window, the illumination window, the treatment tool path, the suction path, the air/water supply path, and the front water-supply path are holes that are penetrated from the proximal end (the end on the side of the operating unit 22) of the rigid member 212 to the distal end thereof, and specifically they have the functions described below.

The observation window is a hole for acquiring optical images inside the subject. Furthermore, the inlet end side of the above-described image guide is inserted into the inside of the observation window. Moreover, the inlet end of the above-described image guide is connected to an objective lens (not illustrated).

The illumination window is a hole for emitting illumination light to inside of the subject. Furthermore, the outlet end side of the above-described light guide is inserted into the inside of the illumination window.

The treatment tool path is a hole for protruding various treatment tools to outside. Furthermore, the above-described treatment tool tube is connected to the treatment tool path.

The suction path is a hole for suctioning fluid, or the like, inside the subject. Furthermore, the above-described suction tube is connected to the suction path.

The air/water supply path is a hole for supplying air or water to the outer surface of the observation window (the objective lens connected to the inlet end of the above-described image guide). Furthermore, the above-described air/water supply tube is connected to the air/water supply path.

The front water-supply path is a hole for supplying water to the inside of the subject. Furthermore, the above-described front water-supply tube is connected to the front water-supply path.

The operating unit 22 is a part that is connected to the proximal end side of the insertion unit 21 and that receives various operations from doctors, and the like. As illustrated in FIG. 1, the operating unit 22 includes a curving knob 221 for performing operation to curve the curved portion 213 and multiple operating members 222 for performing various operations.

Here, various operations that may be performed by the operating members 222 include a suction operation to suction fluid, or the like, inside a subject through the above-described suction path, an air/water supply operation to supply air or water to the outer surface of the observation window through the above-described air/water supply path, a front water-supply operation to supply water to the inside of a subject through the above-described front water-supply path, and the like.

Furthermore, the operating unit 22 is provided with a treatment-tool insertion hole 223 that communicates with the above-described treatment tool tube to insert various treatment tools to the treatment tool tube.

Furthermore, an imaging device (not illustrated) that outputs image signals that correspond to optical images inside the subject and an optical system (not illustrated) that forms optical images, guided by the above-described image guide, on the imaging device are provided inside the operating unit 22. Image signals output from the imaging device are transmitted to the endoscope observation device 4 (the video processor 41) through the above-described signal cable.

The universal cable 23 is a cable whose one end is connected to the operating unit 22 and where the light guide, the signal cables, the suction tube, the air/water supply tube, the front water-supply tube, and the like, which are described above, are provided inside.

The endoscope connector 6 is a connector provided at the other end of the universal cable 23 so as to connect to the ultrasound cable 31 connected to the ultrasound observation device 3 and the endoscope observation device 4 (the video processor 41 and the light source device 42).

Configuration of Endoscope Connector

Next, an explanation is given of a configuration of the endoscope connector 6 with reference to FIGS. 2 to 7.

Hereafter, with reference to the posture of the endoscope connector 6 being connected to the endoscope observation device 4, the upper side in the posture is “up”, the lower side in the posture is “down”, the side close to the endoscope observation device 4 is “front”, the side away from the endoscope observation device 4 is “rear”, the left side viewed from the front side in the posture is “left”, and the right side viewed from the front side in the posture is “right”.

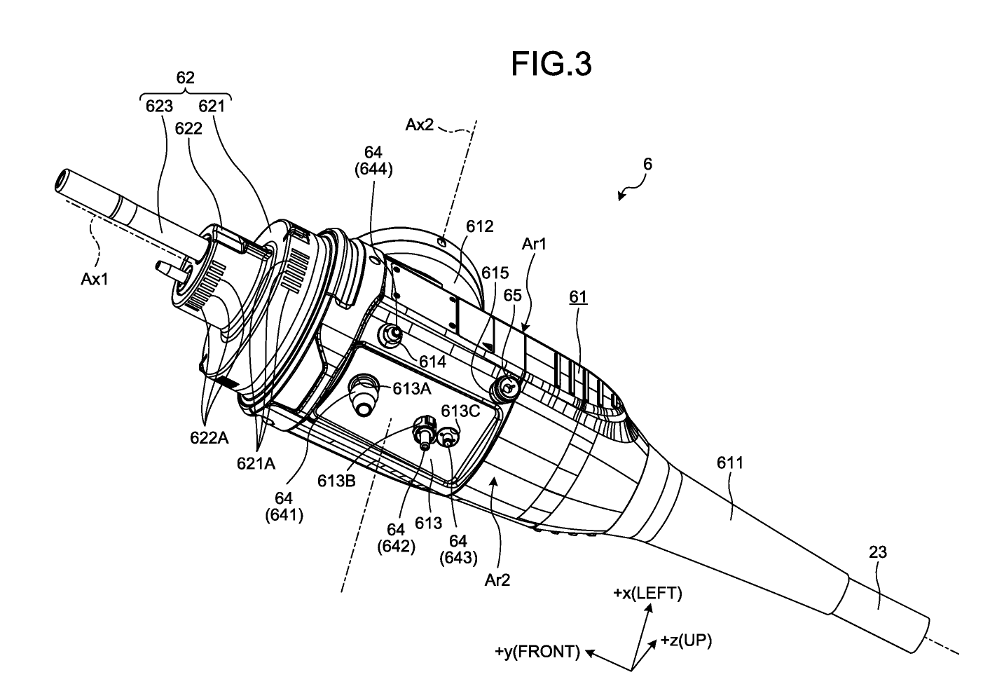

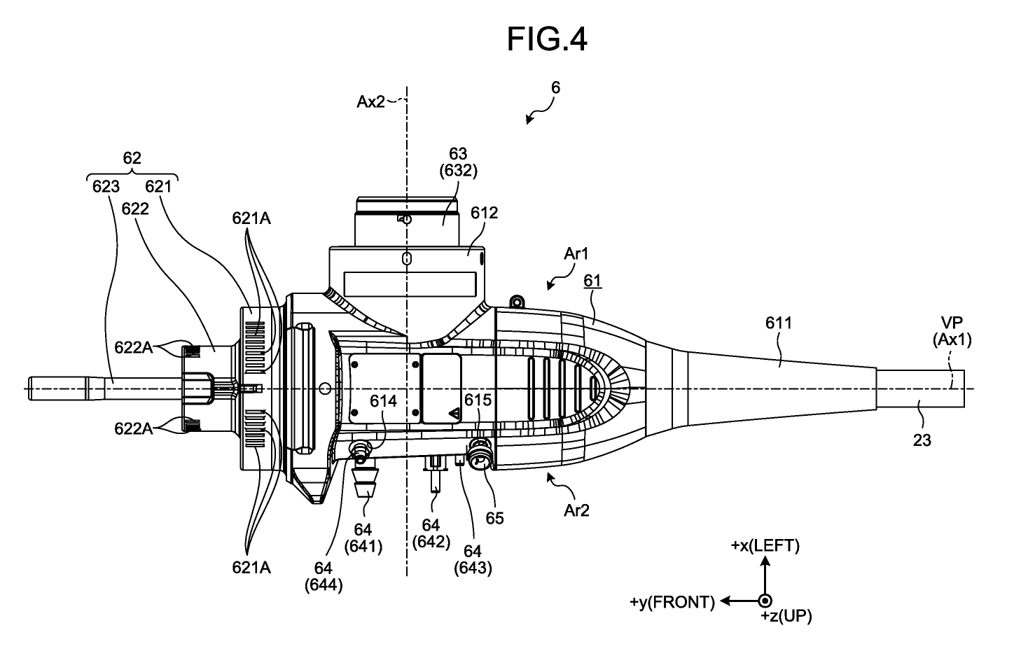

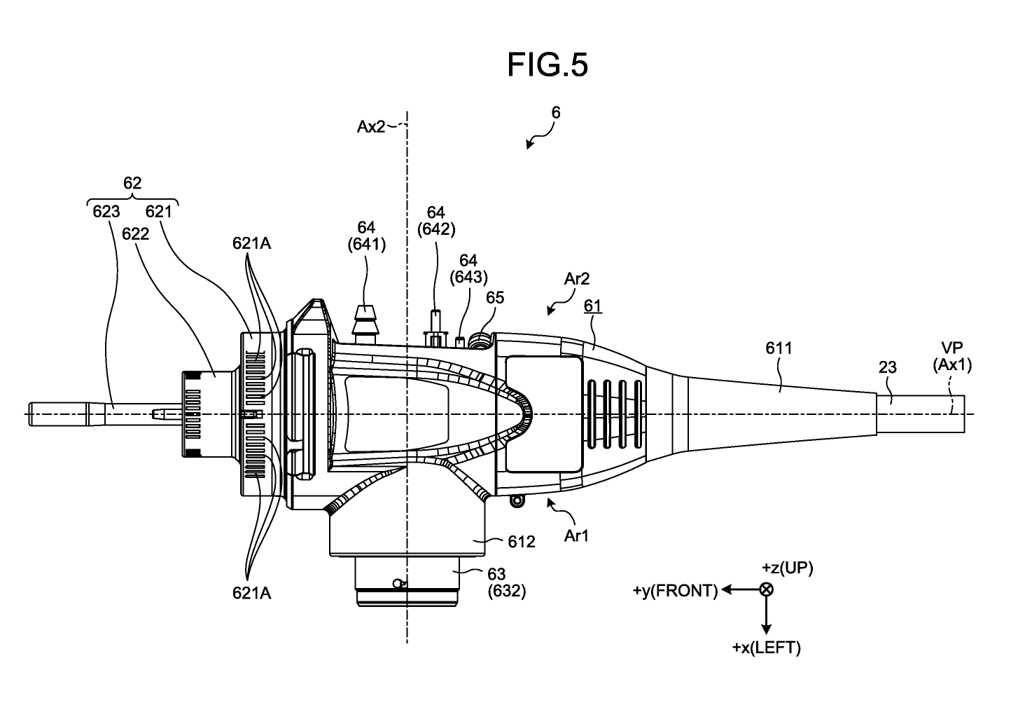

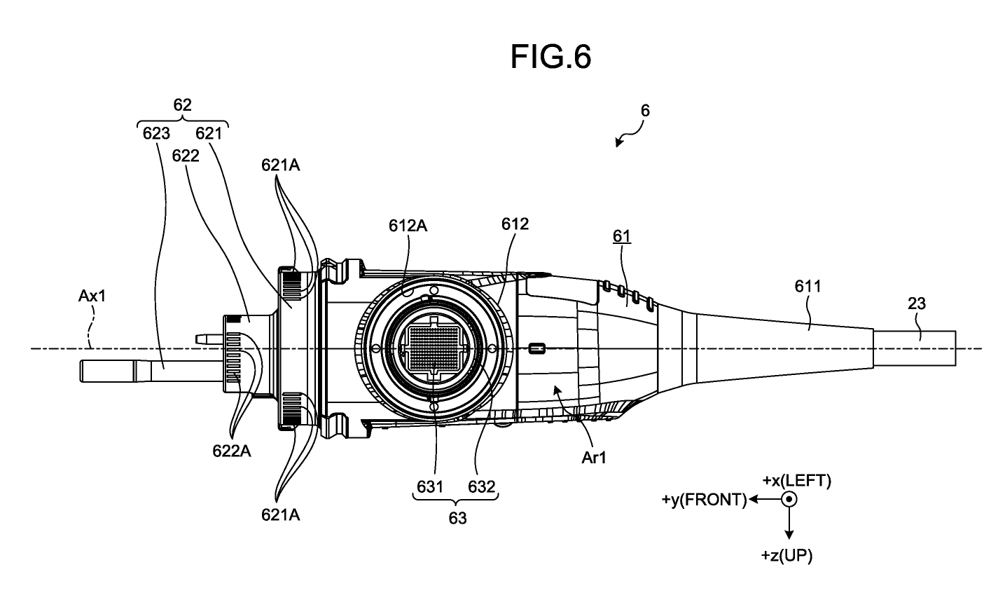

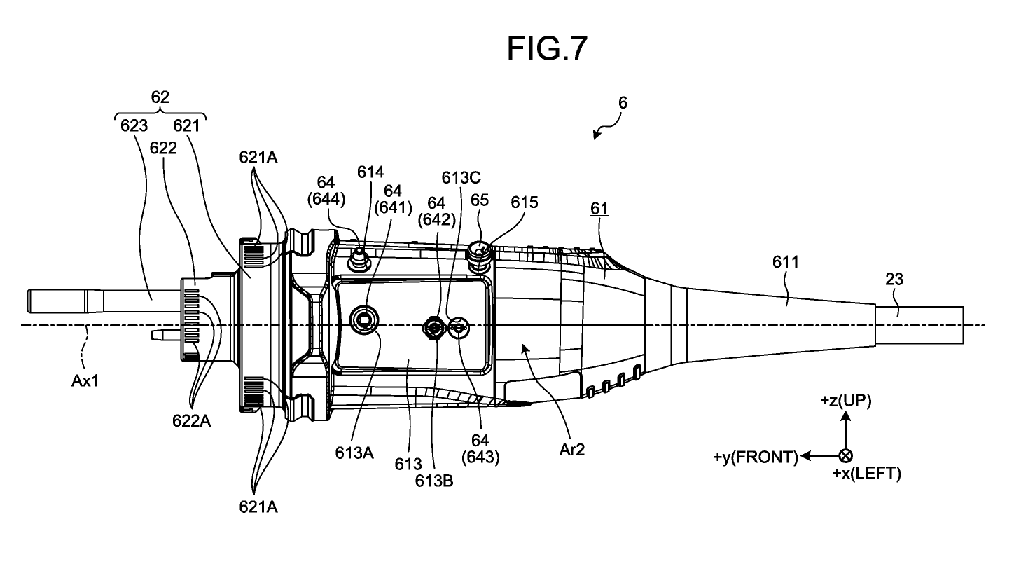

FIG. 2 is a perspective view when the endoscope connector 6 is viewed from the front side and the upper left side. FIG. 3 is a perspective view when the endoscope connector 6 is viewed from the front side and the upper right side. FIG. 4 is a diagram when the endoscope connector 6 is viewed from the upper side. FIG. 5 is a diagram when the endoscope connector 6 is viewed from the lower side. FIG. 6 is a diagram when the endoscope connector 6 is viewed from the left side. FIG. 7 is a diagram when the endoscope connector 6 is viewed from the right side.

Furthermore, FIGS. 2 to 7 illustrate XYZ Cartesian coordinates to identify the above-described “up and down”, “front and rear”, and “right and left” of the endoscope connector 6. Here, the +Z axis direction is an “upper direction” of the endoscope connector 6. The +X axis direction is a “left direction” of the endoscope connector 6. The +Y axis direction is a “front direction” of the endoscope connector 6.

As illustrated in FIGS. 2 to 7, the endoscope connector 6 includes an outer casing 61, a plug unit 62, an ultrasound connector 63 (FIG. 2, FIGS. 4 to 6), four fluid ferrules 64 (FIGS. 2 to 5, FIG. 7), and a terminal 65 (FIGS. 2 to 5, FIG. 7).

As illustrated in FIGS. 2 to 7, the outer casing 61 has a substantially cylindrical shape that extends in a front-back direction (Y axis (equivalent to a “first axis” according to the present disclosure) direction). Furthermore, the universal cable 23 (the light guide, the signal cables, the suction tube, the air/water supply tube, the front water-supply tube, and the like, which are described above) is inserted into the inside of the outer casing 61 through an opening section on the rear side. Furthermore, as illustrated in FIGS. 2 to 7, a fold preventing member 611 is provided on the rear side of the outer casing 61.

Hereafter, as illustrated in FIG. 4 or 5, the side surface of the outer casing 61 is virtually separated into two areas with a virtual plane VP (YZ plane) that includes a central axis Ax1 (the axis parallel to the Y axis) of the outer casing 61 so that one of the two areas is a first area Ar1 and the other area is a second area Ar2.

As illustrated in FIGS. 2 to 6, on the side surface of the outer casing 61, a protruding section 612 is formed on the first area Ar1 such that it is perpendicular to the central axis Ax1 and protrudes in the +X axis direction.

The protruding section 612 communicates with the inside of the outer casing 61 and it is shaped to have a hollow. Furthermore, as illustrated in FIG. 2 or 6, the protruding section 612 is provided with a connector attachment hole 612A that has an opening area positioned on the YZ plane and that communicates with inside or outside of the outer casing 61. The connector attachment hole 612A is equivalent to a first attachment hole according to the present disclosure, and it is a hole to which the ultrasound connector 63 is attached.

Furthermore, as illustrated in FIG. 3 or 7, on the side surface of the outer casing 61, a recess portion 613 is formed on the second area Ar2 such that it is recessed toward the inside of the outer casing 61. The recess portion 613 is formed at a position on the opposite side of the position where the protruding section 612 is formed (the position rotated 180 degrees from the position where the protruding section 612 is formed with the central axis Ax1 as a center). Furthermore, the bottom section of the recess portion 613 is formed to be flat as being substantially parallel to the YZ plane. Moreover, as illustrated in FIG. 3 or 7, first to third ferrule attachment holes 613A to 613C are formed on the bottom section of the recess portion 613. The first to third ferrule attachment holes 613A to 613C are equivalent to second attachment holes according to the present disclosure.

The first ferrule attachment hole 613A is a hole for attaching a suction duct ferrule 641 among the four fluid ferrules 64.

Here, although not illustrated in detail, a suction duct is provided inside the outer casing 61 such that it communicates with outside of the outer casing 61 through the first ferrule attachment hole 613A and extends to the rear side so as to be connected to the above-described suction tube.

Furthermore, in order to ensure a connection space for the above-described suction tube and the above-described suction duct inside the outer casing 61, the above-described first ferrule attachment hole 613A is formed in front of the second and third ferrule attachment holes 613B, 613C. As the first ferrule attachment hole 613A is provided at this position, it is possible to prevent an increase in the length of the outer casing 61 in a front-back direction.

The second and third ferrule attachment holes 613B, 613C are holes for attaching two air/water supply duct ferrules 642, 643, respectively, included in the four fluid ferrules 64.

Here, although not illustrated in detail, air/water supply ducts are provided inside the outer casing 61 such that they communicate with outside of the outer casing 61 through the second and third ferrule attachment holes 613B, 613C, respectively, and extend to the rear side so as to be connected to the above-described air/water supply tube (bifurcated at one end).

Furthermore, as illustrated in FIG. 3, 4, or 7, on the side surface of the outer casing 61, a fourth ferrule attachment hole 614 for attaching a front water-supply duct ferrule 644 included in the four fluid ferrules 64 is formed on an upper side of the recess portion 613 on the second area Ar2.

Here, although not illustrated in detail, a front water-supply duct is provided inside the outer casing 61 such that it communicates with outside of the outer casing 61 through the fourth ferrule attachment hole 614 and extends to the rear side so as to be connected to the above-described front water-supply tube.

Furthermore, as illustrated in FIG. 3, 4, or 7, on the side surface of the outer casing 61, a terminal attachment hole 615 for attaching the terminal 65 is formed on an upper side of the recess portion 613 on the second area Ar2.

The plug unit 62 is a part that is inserted into the endoscope observation device 4 to be connected to the video processor 41 and the light source device 42 and, as illustrated in FIGS. 2 to 7, it is attached to an opening area at the front side of the outer casing 61. As illustrated in FIGS. 2 to 7, the plug unit 62 includes first and second electric connector sections 621, 622, which are waterproof-type electric connectors, and a light guide ferrule 623.

As illustrated in FIGS. 2 to 7, the first electric connector section 621 is located at the rearmost position of the plug unit 62 and it has a cylindrical shape that extends in a front-back direction.

Multiple first electric contact points 621A are provided along a circumferential direction on part of the outer circumference of the first electric connector section 621.

As illustrated in FIGS. 2 to 7, the second electric connector section 622 is integrally formed on the front side of the first electric connector section 621 and it has a cylindrical shape with an outer diameter smaller than the outer diameter of the first electric connector section 621.

Multiple second electric contact points 622A are provided along a circumferential direction on part of the outer circumference of the second electric connector section 622.

The above-described first and second electric contact points 621A, 622A are electrically connected to the above-described signal cables. Furthermore, the first and second electric contact points 621A, 622A are electrically connected to the video processor 41 when the plug unit 62 is inserted into the endoscope observation device 4. That is, the first and second electric contact points 621A, 622A are parts that electrically connect the above-described signal cables and the video processor 41.

The light guide ferrule 623 is attached to the front edge surface of the second electric connector section 622, and it protrudes from the front edge surface in the +Y axis direction.

Furthermore, the inlet end side of the above-described light guide is inserted into the light guide ferrule 623. Furthermore, the light guide ferrule 623 is connected to the light source device 42 when the plug unit 62 is inserted into the endoscope observation device 4. That is, the light guide ferrule 623 is a part that optically connects the above-described light guide and the light source device 42.

The ultrasound connector 63 is a non-waterproof type electric connector that electrically connects the above-described signal cables and the ultrasound cable 31. As illustrated in FIG. 2 or FIGS. 4 to 6, the ultrasound connector 63 includes a terminal 631 (FIG. 2, FIG. 6) that is electrically connected to the above-described signal cables; and a connector section 632 that has a cylindrical shape to cover the terminal 631 and that is connected to a connector at the side of the ultrasound cable 31.

Furthermore, the ultrasound connector 63 is fastened with a screw, or the like, while one end side of the connector section 632 is engaged with the connector attachment hole 612A. When the ultrasound connector 63 is attached to the connector attachment hole 612A in this way, a central axis Ax2 (the axis parallel to the X axis (equivalent to a “second axis” according to the present disclosure)) of the connector section 632 is perpendicular to the central axis Ax1 of the outer casing 61, as illustrated in FIG. 4 or 5. That is, the ultrasound connector 63 extends along the “second axis” according to the present disclosure.

As illustrated in FIGS. 2 to 5 or FIG. 7, the four fluid ferrules 64 include the suction duct ferrule 641 (FIGS. 3 to 5, FIG. 7), the two air/water supply duct ferrules 642, 643 (FIGS. 3 to 5, FIG. 7), and the front water-supply duct ferrule 644 (FIGS. 2 to 4, FIG. 7).

As illustrated in FIGS. 3 to 5 or FIG. 7, the suction duct ferrule 641 is attached to the first ferrule attachment hole 613A such that it protrudes in the −X axis (equivalent to the “second axis” according to the present disclosure) direction from the side surface of the outer casing 61 and it communicates with the suction duct provided inside the outer casing 61. That is, the suction duct ferrule 641 extends along the “second axis” according to the present disclosure. Furthermore, although not illustrated in detail, an external suction device, such as a suction pump, is connected to the suction duct ferrule 641. Furthermore, when a suction operation is conducted on the operating member 222, fluid or the like inside the subject is suctioned from the distal end of the insertion unit 21 to the external suction pump through the inside of the endoscope 2 (the suction path, the suction tube, the suction duct, and the suction duct ferrule 641).

As illustrated in FIGS. 3 to 5 or FIG. 7, the two air/water supply duct ferrules 642, 643 are attached to the second and third ferrule attachment holes 613B, 613C, respectively, such that they protrude in the −X axis direction from the side surface of the outer casing 61 and they communicate with the two air/water supply ducts, respectively, provided inside the outer casing 61. That is, the two air/water supply duct ferrules 642, 643 extend along the “second axis” according to the present disclosure. Furthermore, although not illustrated in detail, an external air/water supply device, such as a water supply pump or an air supply pump, is connected to each of the two air/water supply duct ferrules 642, 643. Furthermore, when an air/water supply operation is performed on the operating member 222, air or water is supplied to the outer surface of the observation window provided in the rigid member 212 from the external air/water supply device through the inside of the endoscope 2 (the air/water supply duct ferrules 642, 643, the air/water supply duct, the air/water supply tube, and the air/water supply path).

That is, the “air/water supply duct ferrule” according to the present disclosure refers to a ferrule for supplying air or water to the outer surface of the observation window through the inside of the endoscope 2.

As illustrated in FIG. 3, 4, or 7, the front water-supply duct ferrule 644 is attached to the fourth ferrule attachment hole 614 such that it protrudes from the side surface of the outer casing 61 to an upper right side and it communicates with the front water-supply duct that is provided inside the outer casing 61. Furthermore, although not illustrated in detail, the front water-supply duct ferrule 644 is connected to an external water supply device, such as a water supply pump. Furthermore, when a front water-supply operation is performed on the operating member 222, water is supplied by the external water supply device from the distal end of the insertion unit 21 to the inside of the subject through the inside of the endoscope 2 (the front water-supply duct ferrule 644, the front water-supply duct, the front water-supply tube, and the front water-supply path).

The terminal 65 returns high-frequency leakage current, which leaks from a treatment tool to the endoscope during endoscopic high-frequency treatment, to a high-frequency cautery power-supply device connected to the treatment tool and, as illustrated in FIG. 3, 4, or 7, it is attached to the terminal attachment hole 615 such that it protrudes from the side surface of the outer casing 61 to an upper right side and it is secured to be in contact with a reinforcing metallic frame (not illustrated) provided inside the outer casing 61 so as to be electrically connected together.

Positions of Four Fluid Ferrules

The above-described four fluid ferrules 64 (the suction duct ferrule 641, the two air/water supply duct ferrules 642, 643, and the front water-supply duct ferrule 644) are located at the following positions when they are attached to the first to fourth ferrule attachment holes 613A to 613C and 614.

Specifically, as illustrated in FIG. 7, when viewed in the −X axis direction, the four fluid ferrules 64 are positioned within the side surface of the outer casing 61. In other words, the four fluid ferrules 64 are positioned between an upper end and a lower end on the side surface of the outer casing 61 when viewed in the −X axis direction, and they do not protrude above the upper end or below the lower end.

In the endoscope connector 6 according to the present embodiment described above, the ultrasound connector 63 is provided on the first area Ar1. Furthermore, the four fluid ferrules 64 are provided on the second area Ar2. Furthermore, when viewed in the direction (the X axis direction) in which the ultrasound connector 63 extends, the four fluid ferrules 64 are positioned within the side surface of the outer casing 61. That is, in the endoscope connector 6, the ultrasound connector 63 and the four fluid ferrules 64 are provided at positions away from each other.

Therefore, the endoscope connector 6 according to the present embodiment has an advantage such that, even if fluid leaks through the four fluid ferrules 64, the ultrasound connector 63 may be prevented from getting wet.

Furthermore, before and after the endoscope 2 is used, the endoscope connector 6 is immersed in a washing tank that contains cleaning liquid to be cleaned while a waterproof cap (not illustrated) is attached to the ultrasound connector 63. Here, when the endoscope connector 6 is immersed in the cleaning liquid, it is in the posture when it is connected to the endoscope observation device 4 (in the posture such that the direction in which the ultrasound connector 63 extends is in a horizontal direction).

In the endoscope connector 6 according to the present embodiment, the four fluid ferrules 64 are positioned within the side surface of the outer casing 61 when viewed in the direction in which the ultrasound connector 63 extends.

Therefore, when the endoscope connector 6 is immersed in cleaning liquid, the four fluid ferrules 64 are prevented from protruding out of the liquid surface so that the four fluid ferrules 64 may be sufficiently cleaned.

Furthermore, in the endoscope connector 6 according to the present embodiment, the suction duct ferrule 641 and the two air/water supply duct ferrules 642, 643 are provided such that they protrude from the side surface of the outer casing 61 in the −X axis direction.

Therefore, the distal ends of the suction duct ferrule 641 and the two air/water supply duct ferrules 642, 643 may be located at positions farthest from the ultrasound connector 63 and, as described above, an advantage may be produced such that “even if fluid leaks, the ultrasound connector 63 may be prevented from getting wet” in a preferred manner.

### Other Embodiments

Although the embodiment for implementing the present disclosure has been described above, the present disclosure is not limited to the above-described embodiment.

According to the above-described embodiment, the endoscope system 1 has both the function to generate ultrasound images and the function to generate endoscope images; however, this is not a limitation, and a configuration may be such that it has only any one of the functions.

According to the above-described embodiment, the fluid ferrules according to the present disclosure include the front water-supply duct ferrule 644; however, this is not a limitation, and a configuration may be such that the front water-supply duct ferrule 644 is omitted.

According to the above-described embodiment, the endoscope system 1 may be an endoscope system that is used in not only medical fields but also industrial fields to observe the inside of subjects, such as mechanical structures.

In the endoscope connector according to the present disclosure, the non-waterproof type electric connector is provided at the above-described first area. Furthermore, the fluid ferrules are provided at the above-described second area. Furthermore, the fluid ferrules are positioned within the side surface of the outer casing when viewed in the direction in which the non-waterproof type electric connector extends (the direction parallel to the above-described second axis). That is, in the endoscope connector, the non-waterproof type electric connector and the fluid ferrules are provided at positions away from each other.

Therefore, the endoscope connector according to the present disclosure has an advantage such that, even if liquid leaks through the fluid ferrule, the non-waterproof type electric connector may be prevented from getting wet.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope, comprising:
an insertion unit adapted to be inserted into a subject;
an operating unit provided at a proximal end side of the insertion unit;
a universal cable whose first end is connected to the operating unit; and
an endoscope connector connected to a second end of the universal cable, and extending along a first axis, the endoscope connector including:
a non-waterproof type ultrasound connector provided in a first region, extending along a second axis perpendicular to a virtual plane including the first axis, and electrically connected to an ultrasound observation device;
a suction duct ferrule provided in a second region, and configured to be connected to a suction device for that suctions fluid inside the subject from a distal end of the insertion unit; and
an air/water supply duct ferrule provided in the second region, and configured to be connected to an air/water supply device that supplies air or water to an observation window of the insertion unit,
wherein the endoscope connector is virtually divided into the first region and the second region by the virtual plane including the first axis.

2. The endoscope according to claim 1, wherein the endoscope connector further includes a front water-supply duct ferrule provided in the second region, and configured to be connected to a water supply device that supplies water into the subject from the distal end of the insertion unit.

3. The endoscope according to claim 1, wherein the suction duct ferrule is located at a position farthest from the non-waterproof type ultrasound connector.

4. The endoscope according to claim 1, wherein the air/water supply duct ferrule is located at a position farthest from the non-waterproof type ultrasound connector.

5. The endoscope according to claim 1, wherein
the endoscope connector further includes a recess portion in the second region, and
the suction duct ferrule and the air/water supply duct
ferrule are provided in the recess portion.
